(19) United States
(12) Patent Application Publication
(10) Pub. No.: US 2004/0199035 A1
(43) Pub. Date: Oct. 7, 2004

(54) Method for synthesising terminal olefins by combining isomerisation metathesis and isomersation transalkylation

(76) Inventors: Jörn Karl, (US); Dag Wiebelhaus, Neustadt (DE); Heiko Maas, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/483,988

(22) PCT Filed: Jul. 24, 2002
(86) PCT No.: PCT/EP02/08253

(30) Foreign Application Priority Data
Jul. 25, 2001 (DE) 101 36 048.7

(51) Int. Cl. C07C 2/00

(52) U.S. Cl. 585/324

(57) ABSTRACT

A process for the targeted preparation of long-chain α-olefins having a narrow molecular weight distribution comprises the following steps:

i) Introduction of a C₄-C₁₀-olefin fraction into an isomerizing metathesis reaction,

ii) Fractionation of the mixture obtained to give

a) a C₂-C₃-olefin fraction,

b) a fraction comprising olefins having the desired number of carbon atoms,

c) a light fraction comprising olefins having a number of carbon atoms ranging from 4 to the integer below the number of carbon atoms of the desired fraction b) and

d) a heavy fraction comprising olefins having a number of carbon atoms above that of the desired fraction b),

iiii) Recirculation of the light fraction c) and, if desired, the heavy fraction d) to the isomerizing metathesis reaction i),

iv) Reaction of the fraction b) with a trialkylaluminum compound in a transalkylation under isomerizing conditions, in which an olefin corresponding to the alkyl radical is liberated and the olefins used add onto the aluminum with isomerization and formation of corresponding alkylaluminum compounds,

v) Reaction of the alkylaluminum compounds formed in iv) with an olefin to liberate the α-olefins corresponding to the alkylaluminum compounds formed in step iv).

The present invention relates to a process for preparing long-chain α-olefins having a narrow molecular weight distribution, which process comprises an isomerizing metathesis and an isomerizing transalkylation as process steps. The process is suitable for the preparation of, preferably, α-olefins having from 6 to 15 carbon atoms. In particular, the process can be used for preparing linear α-olefins having from 8 to 12 carbon atoms, more preferably from 9 to 11 carbon atoms, and α-olefins having from 10 to 15 carbon atoms and a certain degree of branching.

Owing to their carbon-carbon double bond by means of which it is possible to introduce many functional groups, olefins represent the most important class of basic chemicals for the chemical industry. There are a variety of processes for preparing olefins which, as a person skilled in the art will know, are divided into various classes, for example short- and long-chain, linear and branched olefins or olefins having internal and terminal double bonds. Cracking of saturated hydrocarbons is the most frequently employed method of preparing olefins. However, this is primarily suitable for preparing short-chain olefins having up to 4 carbon atoms.

Higher linear α-olefins having from about 6 to 20 carbon atoms constitute a class of olefins which, after further processing, have found a wide variety of uses in the production of laundry detergents, plasticizers and lubricating oils. There are only a limited number of processes for preparing this class of olefins. Among these, dehydration of natural alcohols and cracking of higher paraffins (wax cracking) are unimportant. Most linear α-olefins are produced by transition metal-catalyzed oligomerization of ethylene using the Ziegler process or the SHOP process of Shell, by means of which highly linear olefin fractions having α-olefin contents of >95% can be obtained. Catalysts used in the Ziegler process are aluminum alkyls, while in the SHOP process, phosphine-modified nickel complexes are employed as active species in the oligomerization reaction. The length distribution of the carbon chain conforms to the Schulz-Flory distribution with a high proportion of short-chain α-olefins. The proportion of a particular α-olefin deceases exponentially with increasing number of carbon atoms.

To be able to make use of the short- and long-chain olefins remaining after separating off the desired C₆-C₂₀-olefins, these are isomerized in the SHOP process to form olefins having an internal double bond and the resulting mixture is subjected to a metathesis reaction. This forms, for example, olefins having from 7 to 20 carbon atoms and internal double bonds.

U.S. Pat. No. 3,491,163 discloses a process for building up olefins which does not rely on a transition metal-catalyzed oligomerization. Propylene as starting olefin is firstly subjected to a metathesis reaction. The resulting C₄ fraction which has been freed of lighter and heavier olefins is then used as starting material in an isomerizing metathesis reaction after which the resulting C₅-C₆-olefin fraction is separated from the lighter and heavier isomers and is once again subjected to an isomerizing metathesis. The desired product obtained in this way, namely C₇-C₁₀-olefins, is isomerized, subsequently freed of lighter and heavier fractions and subsequently subjected to isomerization and metathesis for a final time. The light and heavy olefins separated off in each case are recirculated and reused in the reaction or, in the case of ethylene, used in a reaction for building up the molecular weights. A mixture of C₁₁-C₁₅-olefins having an internal double bond is obtained.

In contrast, the process described in WO 97/34854 allows the preparation of linear α-olefins. In this process, an olefin mixture comprising olefins having an internal double bond and from 6 to 30 carbon atoms is subjected to metathesis under nonequilibrium conditions. The product formed is separated into a low-boiling olefin fraction and a higher-boiling olefin fraction. Both fractions consist of internal olefins. The higher-boiling fraction is subsequently subjected to metathesis with ethylene (ethenolysis) in which the abovementioned α-olefins are formed. However, no buildup reaction to form olefins takes place in the process described, but only conversion of an olefin fraction having an internal double bond into an olefin fraction having terminal double bonds.

Although the transition metal-catalyzed buildup reaction and the process disclosed in U.S. Pat. No. 3,491,163 give high linear α-olefin fractions having the desired numbers of carbon atoms in very good or at least satisfactory yields, these processes of the prior art have the disadvantage that only ethylene or propylene can be used as starting olefins. However, the preparation of ethylene and propylene by cracking produces, depending on the cracking conditions and especially on the choice of starting material, a variable amount of C₄₊-olefins (olefins having >3 carbon atoms). While propylene is a very sought-after raw material, for example for the preparation of polypropylene, the C₄₊-olefins are frequently obtained in amounts which significantly exceed demand.

Furthermore, olefin mixtures comprising linear α-olefins having up to 15 carbon atoms, in particular from 10 to 15 carbon atoms, together with a variable proportion of branched α-olefins containing a number of carbon atoms in this range have recently become economically important. Such olefin mixtures are used to prepare surfactants, e.g. alkylbenzenes and surfactant alcohols which are employed as raw materials for laundry detergents.

It is an object of the present invention to provide a process by means of which lower olefins, in particular those obtained in a cracking process, can be converted into α-olefins having from 6 to 20 carbon atoms. The process should preferably produce linear α-olefins having from 8 to 12 carbon atoms, in particular from 9 to 11 carbon atoms, and branched α-olefins having from 10 to 15 carbon atoms or mixtures of these branched olefins with linear α-olefins having numbers of carbon atoms in the same range.

The patent application DE 100 41 345.5 by the present applicant, which is not a prior publication, discloses a process for preparing long-chain α-olefins having a narrow molecular weight distribution, which comprises the following process steps:

i) Introduction of a linear C₄-C₁₀-olefin fraction into an isomerizing metathesis reaction,

ii) Fractionation of the mixture obtained to give

a) a C₂-C₃-olefin fraction,

b) a fraction comprising olefins having the desired number of carbon atoms,

c) a light fraction comprising olefins having a number of carbon atoms ranging from 4 to the integer below the number of carbon atoms of the desired fraction b) and

d) a heavy fraction comprising olefins having a number of carbon atoms above that of the desired fraction b),

iii) Recirculation of the light fraction c) and, if desired, the heavy fraction d) to the isomerizing metathesis reaction i),

iv) Introduction of the fraction b) into an ethenolysis reaction,

v) Isolation of the α-olefin fraction prepared in iv).

The present invention relates to a process which likewise achieves the object described above.

The present invention provides a process for preparing long-chain α-olefins having a narrow molecular weight distribution, which comprises the following process steps:

i) Introduction of a C₄-C₁₀-olefin fraction into an isomerizing metathesis reaction,

ii) Fractionation of the mixture obtained to give

a) a C₂-C₃-olefin fraction,

b) a fraction comprising olefins having the desired number of carbon atoms,

c) a light fraction comprising olefins having a number of carbon atoms ranging from 4 to the integer below the number of carbon atoms of the desired fraction b) and

d) a heavy fraction comprising olefins having a number of carbon atoms above that of the desired fraction b),

iiii) Recirculation of the light fraction c) and, if desired, the heavy fraction d) to the isomerizing metathesis reaction i),

iv) Reaction of the fraction b) with a trialkylaluminum compound in a transalkylation under isomerizing conditions, in which an olefin corresponding to the alkyl radical is liberated and the olefins used add onto the aluminum with isomerization and formation of corresponding alkylaluminum compounds,

v) Reaction of the alkylaluminum compounds formed in iv) with an olefin to liberate the α-olefins corresponding to the alkylaluminum compounds formed in step iv).

Appropriate routing of the circulating strews and stealthful setting of suitable reaction conditions enable the metathesis reaction i) to be carried out in such a way that a high proportion of higher olefins is present in the product obtained. It is thus possible to carry out a build up of the carbon chain by means of the metathesis reaction.

Suitable feed mixtures for the metathesis reaction i) are, firstly, short-chain linear olefins having from 4 to 10 carbon atoms, which can originate, for instance, from steam crackers or FCC plants. For example, such fractions comprise cis/trans-butenes, cis/trans-pentenes and cis/trans-hexenes having the double bond in different positions. It is also possible to use olefins having the desired number of carbon atoms or olefin mixtures having numbers of carbon atoms in the desired range which originate from the Fischer-Tropsch process. Preference is given to using a C₄-C₆-olefin mixture. C₄-olefins are particularly useful as starting materials.

These are obtained, inter alia, in various cracking processes such as steam cracking or fluid catalytic cracking as C₄ fraction. As an alternative, it is possible to use butene mixtures as are obtained in the dehydrogenation of butanes or by dimerization of ethene. Butanes present in the C₄ fraction behave as inert. Dienes, allenes or enynes present in the mixture used are removed by means of customary methods such as extraction or selective hydrogenation.

The butene content of the C₄ fraction which is preferably used in the process is from 1 to 100% by weight, preferably from 60 to 90% by weight. This butene content is based on 1-butene and 2-butene.

Since olefin-containing C4-hydrocarbon mixture are available at low cost, the use of these mixtures improves the value added to by-products from a steam cracker. Furthermore, products having a higher added value are obtained.

Preference is given to using a C4 fraction obtained in steam cracking or fluid catalytic cracking or in the dehydrogenation of butane.

As C4 fraction, particular preference is given to using raffinate II, with the C4 stream being freed of interfering impurities, in particular oxygen compounds, by appropriate treatment over adsorbent guard beds, preferably over high surface area aluminum oxides and/or molecular sieves. Raffinate II is obtained from the C4 fraction by firstly extracting butadiene and/or subjecting it to a selective hydrogenation. Removal of isobutene then gives raffinate II. Suitable processes are disclosed in DE 100 13 253.7 by the present applicant.

A second group of olefins which can be used in the metathesis i) are branched C₄-C₁₀-olefins. This variant is of interest in cases where branched α-olefins or mixtures comprising such branched olefins together with linear α-olefins are to be prepared. These branched olefins or the mixtures in which they are present are, for example, preferably used for the preparation of alkylbenzenes or alkylbenzenesulfonates.

Another possible way of obtaining branched olefins is to carry out the metathesis reaction under conditions under which structural isomerization of the hydrocarbon chain occurs. This is described in more detail below.

The relative amount of branched olefins or the degrees of branching necessary for a given application are known to those skilled in the art and can be set in a manner known per se, for example by choice of starting materials and/or reaction parameters.

It is advisable to free the olefins used of impurities by appropriate treatment over adsorbent guard beds, preferably over aluminum oxides having a large surface area or molecular sieves. Other upstream purification steps are known to those skilled in the art.

The metathesis reaction i) is carried out over a catalyst which catalyzes both the metathesis reaction and the double bond isomerization of the olefins formed. It is possible for a metathesis catalyst and an isomerization catalyst to be present separately in the reactor. In an alternative embodiment of the present invention, the metathesis and the isomerization reaction can be carried out in separate reactors of which one contains the isomerization catalyst and the other the metathesis catalyst. In this case, it is possible to carry out firstly the metathesis and then the isomerization, but the metathesis can also follow the isomerization.

The metathesis catalyst comprises a compound of a metal of Group VIb, VIIb or VIII of the Periodic Table of the Elements. The metathesis catalyst preferably comprises an oxide of a metal of Group VIb or VIIb of the Periodic Table of the Elements. In particular, the metathesis catalyst is selected from the group consisting of Re₂O₇, WO₃ and MoO₃.

The isomerization catalyst comprises a metal of Group Ia, IIa, IIIb, IVb, Vb or VIII of the Periodic Table of the Elements or a compound thereof. The isomerization catalyst is preferably selected from the group consisting of Re₂O₇, RuO₂, NiO, MgO, Na and K₂CO₃. It is also possible to use homogeneous isomerization catalysts, e.g. Ni(0) with aluminum alkyls or metallocenes of Ti, Zr or Hf.

Preference is given to using a catalyst which is active both as a metathesis catalyst and as an isomerization catalyst. Such a catalyst comprises a combination of the abovementioned catalyst components, i.e. comprises a compound of a metal of Group VIb, VIIb or VIII for catalyzing the metathesis and an element of Group Ia, IIa, IIIb, IVb, Vb or VIII of the Periodic Table of the Elements for catalyzing the isomerization reaction. Preferred and particularly preferred mixed catalysts comprise at least one from each group of the compounds mentioned above as preferred and particularly preferred.

The catalysts are generally supported on the customary materials known to those skilled in the art. Examples of suitable materials include SiO₂, γ-Al₂O₃, MgO, B₂O₃ or mixtures of these materials, for example γ-Al₂O₃/B₂O₃/SiO₂.

The isomerizing metathesis i) is generally carried out at from 20 to 450° C. If the preparation of linear α-olefins is desired, the temperature in the metathesis reaction i) is preferably in the range from 40 to 100° C. In the case of the preparation of branched α-olefins, the metathesis i) is preferably carried out at from 80 to 150° C. The pressures employed are from 1 to 60 bar, preferably from 10 to 45 bar, in particular from 30 to 35 bar.

Setting of reaction parameters known to those skilled in the art allows the isomerizing metathesis to be carried out so that a high proportion of olefins having a number of carbon atoms in the desired range is obtained. These reaction parameters include, for example, the number of carbon atoms in the feed olefins, the choice of catalysts, the reaction temperature, the residence time, the proportion of product formed which is discharged and also the composition and degree of recirculation of the olefin fraction obtained after the isomerizing metathesis and the subsequent fractionation.

As stated, the metathesis reaction i) can be carried out in such a way that branching of the olefins used occurs. This branching can be achieved, for example, by the use of a catalyst having acid centers and/or selection of a sufficiently high reaction temperature.

In a particularly preferred embodiment of the process of the present invention, a linear C₄ fraction is used in the metathesis reaction i) and subjected to metathesis under conditions under which branching occurs. This gives, as fraction b), a C₁₀-C₁₅-olefin fraction having at least a proportion of branched olefins which is then converted into a C₁₀-C₁₅-α-olefin fraction in step iv). As an alternative thereto, a C₄-C₁₀ fraction of branched olefins can be used as starting material in the reaction.

In a further particularly preferred embodiment of the present invention, a linear C₄ fraction is used in the isomerizing metathesis reaction i), and a linear C₈-C₁₂-olefin fraction is obtained as fraction b) and this is converted in step iv) into a linear C₈-C₁₂-α-olefin fraction. In particular, a C₉-C₁₁-olefin fraction is obtained in b) and this is converted in iv) to a C₉-C₁₁-α-olefin fraction.

The isomerizing metathesis reaction i) can be carried out continuously or batchwise. Deactivation of the catalyst system is frequently observed after a certain time. This can be remedied by regeneration of the catalysts, generally by heating in an oxygen-containing stream of nitrogen to burn off the organic deposits.

The product mixture obtained after the isomerizing metathesis i) is then fractionated by customary methods, for example by distillation. The desired olefin fraction b) which is subsequently used in the isomerizing transalkylation or else, if desired, processed further to give useful products can be obtained in this way. This desired fraction is preferably the C₈-C₁₂ fraction in the case of linear olefins or the C₁₀-C₁₅ fraction in the case of branched olefins.

In addition, a low-boiling fraction a) comprising the C₂- and C₃-olefins is obtained. These are separated off and processed further by customary methods.

A light olefin fraction c) comprising the olefins whose number of carbon atoms ranges from 4 to the integer below the number of carbon atoms of the desired olefin fraction is also isolated. This fraction is returned to the isomerizing metathesis. This light fraction is preferably the C₄-C₇-olefin fraction.

Finally, a heavy olefin fraction d) comprising olefins whose number of carbon atoms is above the number of carbon atoms of the desired olefin fraction is also obtained. This fraction, too, can be returned to the isomerizing metathesis reaction if desired, or, as an alternative, all or some of the fraction b) can be introduced into the transalkylation reaction iv). The heavy fraction is preferably made up of C₁₃₊ olefins in the case of the preparation of linear olefins or is the C₁₆₊ fraction in the case of the preparation of branched olefins.

All the olefin fractions a), b), c) and d) can be used as such in some applications in which olefins having internal double bonds are as suitable or more suitable than α-olefins, sometimes also more advantageous than α-olefins because of their lower price. Examples of the use of the desired fraction b) include, in particular, C₁₁-C₁₅-olefins after hydroformylation and alkoxylation as laundry detergents and cleaners, C₁₀-C₁₄-olefins after conversion into alkylbenzenes or alkylbenzene sulfonates as laundry detergents and cleaners, and C₆-C₁₀-olefins after hydroformylation and hydrogenation to give plasticizer alcohols.

The fraction b), which comprises olefins having the desired chain length, can thus also, if desired, be taken off without further functionalization in step iv) of the synthetic sequence and processed further to give product.

The desired olefin fraction b) is preferably subjected according to the present invention to a transalkylation in step iv).

For the purposes of the present invention, transalkylation is the reaction of an internal olefin with a trialkylaluminum compound under isomerizing conditions. The internal olefin undergoes a rearrangement with double bond isomerization to give a mixture of internal and terminal olefins, with only the terminal olefins reacting to form an aluminum alkyl. The reaction liberates an olefin corresponding to the alkyl radical which was previously bound to the aluminum.

Subsequent to step iv), the trialkylaluminum compound formed is reacted with an α-olefin, preferably a lower olefin. This forms, by displacement, the desired α-olefin or mixtures thereof. The olefins formed correspond to the alkyl radicals which were formed in iv) after isomerization of the internal olefins and addition onto the trialkylaluminum compound.

In a preferred embodiment of the present invention, the olefin which is liberated in the reaction of the trialkylaluminum compound with the linear, internal olefin is isolated and reacted further in step v) with the trialkylaluminum compound formed to liberate the desired α-olefins.

In all variants of the process of the present invention, preference is given to the olefin liberated in the transalkylation being removed continuously from the reactor.

The transalkylation is preferably carried out by the method described in the patent applications EP-A 505 834 and EP-A 525 760. Here, a linear, internal olefin having from 4 to 30 carbon atoms or a mixture of such olefins having internal double bonds is reacted with a trialkylaluminum compound in a molar ratio of linear olefins having internal double bonds to trialkylaluminum of from 1:1 to not more than 50:1. The reaction occurs in the presence of a catalytic amount of a nickel-containing isomerization catalyst which effects the isomerization of the internal olefinic double bond to produce at least a small amount of linear α-olefin. The alkyl groups are subsequently displaced from the trialkylaluminum to form a new alkylaluminum compound in which at least one of the alkyl groups bound to the aluminum is a linear alkyl derived from the linear α-olefin in question. The alkylaluminum compound is subsequently reacted with a 1-olefin, in the presence or absence of a displacement catalyst, to displace the linear alkyl from the alkylaluminum compound and produce a free, linear α-olefin. The isomerization catalyst is selected from among nickel(II) salts, nickel(II) carboxylates, nickel(II) acetonates and nickel(0) complexes which may be stabilized by a trivalent phosphorous ligand. In another embodiment, the isomerization catalyst is selected from the group consisting of bis(1,5-cyclooctadiene)nickel, nickel acetate, nickel naphthenate, nickel octanoate, nickel-2-ethylhexanoate and nickel chloride.

The transalkylation processes described in the patent applications EP-A 505 834 and EP-A 525 760 form an integral part of the present invention and are hereby incorporated by reference.

The transalkylation reaction can also be carried out according to other variants with which a person skilled in the art will be familiar or have access to. In respect of the present invention, a particularly important variant is that in which not linear but branched olefins are used. In a further important variant, it is possible to use isomerization catalysts in which no Ni or Ni compound is present.

The aluminum alkyls used in the transalkylation are known to those skilled in the art. They are chosen on the basis of availability or, for example, the way in which the reaction is carried out. Examples of these compounds include triethylaluminum, tripropylaluminum, tri-n-butylaluminum and triisobutylaluminum. Preference is given to using tripropylaluminum or triethylaluminum.

In a variant of the invention, the fraction d) comprising the long-chain olefins, or part thereof, is introduced into the transalkylation reaction.

Linear α-olefins having from 8 to 12 carbon atoms are of interest for the preparation of poly-alpha-olefins, with degrees of polymerization n of 3-8 and 10-50 being of particular interest. In particular, decene is of interest for the preparation of poly-alpha-olefins in which n=3-8.

C₁₀-C₁₄-α-olefins are, after hydroformylation, important for the preparation of surfactant alcohols and are also important for the preparation of alkylbenzenes (by reaction with benzene), with use being made of both exclusively linear C₁₀-C₁₄-α-olefins and olefins which have a certain proportion of branched olefins in addition to linear olefins.

Linear C₆-C₁₀-α-olefins are converted, after hydroformylation, into plasticizer alcohols; linear C₆-C₈-α-olefins are preferably employed in LLDPE (linear low density polyethylene).

The invention is illustrated by the examples below:

### EXAMPLE 1

70 g of 3-hexene (96%) were stirred in the presence of 20 g of 10% Re₂O₇/Al₂O₃ (calcined at 550° C. in a stream of air for 12 hours, cooled under N₂) at 150° C. without maintenance of pressure in a 270 ml autoclave for 24 hours. After cooling, 35 g of oligomer mixture (58% based on weight introduced) were left in the autoclave (distribution: C₅: 3.7%, C₆: 9.0%, C₇: 9.7%, C₈: 11.0%, C₉: 12.0%, C₁₀: 11.5%, C₁₁: 9.0%, C₁₂ 8.0%, C₁₃: 6.0%, C₁₄: 4.0, C₁₅: 3.1%, C₁₆: 1.7%, C_{17++ 2.0}%). The proportion of C₈₋₁₂ products was 52%, the proportion of C₁₂₊ products was 25% and the degree of branching (determined by means of hydrogenating GC) was 20%. The C₈-C₁₂-olefin mixture is subsequently subjected according to the present invention to transalkylation.

### EXAMPLE 2

70 g of 3-hexene (96%) were stirred in the presence of 20 g of 10% Re₂O₇/Al₂O₃ (calcined at 550° C. in a stream of air for 12 hours, cooled under N₂) at 200° C. without maintenance of pressure in a 270 ml autoclave for 10 hours. After cooling, 20 g of oligomer mixture (29% based on weight introduced) were left in the autoclave (distribution: C₅: 7.5%, C₆: 70.4%, C₇: 7.4%, C₈: 5.1%, C₉: 3.8%, C₁₀: 2.8%, C₁₁: 1.9%, C₁₂ 1.3%, C₁₃₊: 2.5%). The proportion of C₈₋₁₂ products was 15%, the proportion of C₁₂₊ products was 3.8% and the degree of branching (determined by means of hydrogenating GC) was 20%. The C₈-C₁₂-olefin mixture is subsequently subjected according to the present invention to transalkylation.

### EXAMPLE 3

70 g of 3-hexene (96%) were stirred in the presence of 20 g of 10% Re₂O₇/Al₂O₃ (calcined at 550° C. in a stream of air for 12 hours, cooled under N₂) at 130° C. without maintenance of pressure in a 270 ml autoclave for 10 hours. After cooling, 36 g of oligomer mixture (51% based on weight introduced) were left in the autoclave (distribution: C₅: 15.1%, C₆: 30.7%, C₇: 25.3%, C₈: 16.1%, C₉: 7.3%, C₁₀: 3.6%, C₁₁: 1.7%, C₁₂₊ 0.8%). The degree of branching was 8.3%. The C₈-C₁₂-olefin mixture is subsequently subjected according to the present invention to transalkylation.

### EXAMPLE 4

70 g of 3-hexene (96%) were stirred in the presence of 10 g of 10% Re₂O₇/Al₂O₃ (calcined at 550° C. in a stream of air for 12 hours, cooled under N₂) and 10 g of MgO at 150° C. without maintenance of pressure in a 270 ml autoclave for 10 hours. After cooling, 40 g of oligomer mixture (57% based on weight introduced) were left in the autoclave (distribution: C₅: 14.2%, C₆: 21.8%, C₇: 18.7%, C₈: 15.6%, C₉: 11.6%, C₁₀: 8.0%, C₁₁: 4.9%, C₁₂ 2.7%, C₁₃₊: 2.3%). The proportion of C₈₋₁₂ products was 42%, the proportion of C₁₂₊ products was 5% and the degree of branching (determined by means of hydrogenating GC) was 12%. The C₈-C₁₂-olefin mixture is subsequently subjected according to the present invention to transalkylation.

### EXAMPLE 5

In a 270 ml autoclave, 77 g of raffinate II were depressurized from 36 to 4 bar at 150° C. in the presence of 20 g of 10% Re₂O₇/Al₂O₃ (calcined at 550° C. in a stream of air for 12 hours, cooled under N₂) over a period of 50 hours. After cooling, 7 g of oligomer mixture (10% based on weight introduced) remained in the autoclave (C₄₋₇: 40%, C₈₋₁₁: 28%, C₁₂₊: 32%, high degree of branching). The C₈-C₁₂-olefin mixture is subsequently subjected according to the present invention to transalkylation.

We claim:

1. A process for preparing long-chain α-olefins having a narrow molecular weight distribution, which comprises the following process steps:
i) Introduction of a C₄-C₁₀-olefin fraction into an isomerizing metathesis reaction,
ii) Fractionation of the mixture obtained to give
a) a C₂-C₃-olefin fraction,
b) a fraction comprising olefins having the desired number of carbon atoms,
c) a light fraction comprising olefins having a number of carbon atoms ranging from 4 to the integer below the number of carbon atoms of the desired fraction b) and
d) a heavy fraction comprising olefins having a number of carbon atoms above that of the desired fraction b),
iiii) Recirculation of the light fraction c) and, if desired, the heavy fraction d) to the isomerizing metathesis reaction i),
iv) Reaction of the fraction b) with a trialkylaluminum compound in a transalkylation under isomerizing conditions, in which an olefin corresponding to the alkyl radical is liberated and the olefins used add onto the aluminum with isomerization and formation of corresponding alkylaluminum compounds,
v) Reaction of the alkylaluminum compounds formed in iv) with an olefin to liberate the α-olefins corresponding to the alkylaluminum compounds formed in step iv).

2. A process as claimed in claim 1, wherein a metathesis catalyst and an isomerization catalyst are present in step i) and the metathesis catalyst comprises at least one compound selected from among compounds of metals of Groups VIb, VIIb and VIII of the Periodic Table of the Elements and the isomerization catalyst comprises at least one compound of a metal selected from the group consisting of metals of Groups Ia, IIa, IIIb, IVb, Vb and VIII of the Periodic Table of the Elements.

3. A process as claimed in claim 2, wherein the metathesis catalyst comprises an oxide of a metal of Group VIb or VIIb of the Periodic Table of the Elements, in particular Re₂O₇, WO₃ and/or MoO₃, and the isomerization catalyst comprises Re₂O₇, RuO₂, NiO, MgO, Na and/or K₂CO₃.

4. A process as claimed in claim 2 or 3, wherein a catalyst which is active both as metathesis catalyst and as isomerization catalyst and comprises at least one compound from each group of compounds set forth in claim 2 and 3 for the metathesis catalyst and the isomerization catalyst is used.

5. A process as claimed in any of claims 1 to 4, wherein a linear C₄-C₆-olefin fraction, preferably a linear C₄-olefin fraction, more preferably a C₄-olefin fraction from a butene-containing stream, in particular raffinate II, is used in the isomerizing metathesis reaction i).

6. A process as claimed in any of claims 1 to 5, wherein the fraction b) is a linear C₈-C₁₂-olefin fraction, in particular a linear C₉-C₁₁-olefin fraction.

7. A process as claimed in any of claims 1 to 4, wherein a linear C₄-olefin fraction is used in the isomerizing metathesis reaction i) and this reaction is carried out under conditions under which branching of the hydrocarbon chain of the olefin occurs, and the fraction b) is a C₁₀-C₁₅-olefin fraction which comprises at least a proportion of branched olefins.

8. A process as claimed in any of claims 1 to 4, wherein a C₄-C₁₀-olefin fraction comprising at least a proportion of branched olefins is used in the metathesis reaction i) and the fraction b) is a C₁₀-C₁₅-olefin fraction which comprises at least a proportion of branched olefins.

9. A process as claimed in any of claims 1 to 4 and 7 and 8, wherein the isomerizing metathesis i) is carried out at from 20 to 450° C., preferably from 40 to 100° C., and pressures of from 1 to 60 bar, preferably from 10 to 45 bar, in particular from 30 to 35 bar.

10. A process as claimed in any of claims 1 to 8, wherein the isomerizing metathesis i) is carried out at from 20 to 450° C., preferably from 80 to 150° C., and pressures of from 1 to 60 bar, preferably from 10 to 45 bar, in particular from 30 to 35 bar.

11. A process as claimed in any of claims 1 to 10, wherein the olefin liberated in the transalkylation step iv) is removed continuously from the reactor and/or is used for liberation of the α-olefins in step v).

12. A process as claimed in any of claims 1 to 11, wherein the aluminum alkyl used is a trialkylaluminum compound having C₂-C₁₀-alkyl radicals, preferably tripropylaluminum or triethylaluminum.

13. The use of a C₈-C₁₂-olefin mixture, preferably 1-decene, prepared by a process as claimed in any of claims 1 to 12 for the preparation of poly-alpha-olefins, in particular poly-alpha-olefins having degrees of polymerization of from 3 to 8 and from 10 to 50, for the preparation of surfactant alcohols, alkylbenzenes, plasticizer alcohols and as comonomer in the preparation of LLDPE.

14. A process for preparing long-chain internal olefins having a narrow molecular weight distribution, which comprises the following process steps:
ia) Introduction of a C₄-C₁₀-olefin fraction into an isomerizing metathesis reaction,
iia) Fractionation of the mixture obtained to give
a) a C₂-C₃-olefin fraction,
b) a fraction comprising olefins having the desired number of carbon atoms,
c) a light fraction comprising olefins having a number of carbon atoms ranging from 4 to the integer below the number of carbon atoms of the desired fraction b) and
d) a heavy fraction comprising olefins having a number of carbon atoms above that of the desired fraction b),
iiia) Recirculation of the light fraction c) and, if desired, the heavy fraction d) to the isomerizing metathesis reaction i),
iva) Isolation of the fraction b).
